(19) United States
(12) Patent Application Publication
(10) Pub. No.: US 2008/0287384 A1
(43) Pub. Date: Nov. 20, 2008

(54) Methods of Identifying Agents Having Antiangiogenic Activity

(76) Inventors: Marsha Rosner, Chicago, IL (US); Wei-Jen Tang, Chicago, IL (US); Jia Hong, Hoffman Estates, IL (US)

Correspondence Address: MICHAEL BEST & FRIEDRICH LLP, ONE SOUTH PINCKNEY STREET, P O BOX 1806, MADISON, WI, 53701, US

(21) Appl. No.: 12/067,356

(22) PCT Filed: Sep. 19, 2006
(86) PCT No.: PCT/US06/36359

§ 371 Date: Apr. 4, 2008

Related U.S. Application Data
(60) Provisional application No. 60/718,416, filed on Sep. 19, 2005.

(51) Int. Cl. A61K 31/7088; C12Q 1/02; C12N 5/02; C12N 15/00; A61K 31/70

(52) U.S. Cl. 514/44; 435/29; 435/375; 435/455

(57) ABSTRACT

Provided are assays and methods of identifying antiangiogenic agents including contacting an endothelial cell with a putative antiangiogenic agent and assaying for activation of Rap-1 in the endothelial cell. Also provided are methods of inhibiting angiogenesis and treating conditions associated with improper angiogenesis. Compositions comprising activators of Rap-1 and methods of activating the Rap-1 signaling pathway are also provided. Methods of inhibiting chemotaxis and angiogenesis by contacting cells with Rho inhibitors are provided.

### CROSS-REFERENCE TO RELATED APPLICATIONS

This application claims the benefit of U.S. provisional application No. 60/718,416, filed on Sep. 19, 2005. The provisional application is incorporated herein by reference in its entirety.

### STATEMENT REGARDING FEDERALLY SPONSORED RESEARCH

This invention was made with United States government support awarded by the National Institutes of Health, under grant numbers NS-033858, HL-07605, GM-53459 and GM-62548. The United States has certain rights in this invention.

### INTRODUCTION

Angiogenesis, or neovascularization, is the process by which new blood vessels are formed from pre-existing vasculature. The process is normally tightly regulated and essential to a variety of functions including embryonic development and tissue regeneration. Abnormal angiogenesis has been linked to conditions such as blindness, rheumatoid arthritis, AIDS complications, psoriasis, heart disease and cancer. Unregulated angiogenesis can either directly cause a particular disease or exacerbate an existing pathological condition. For example, ocular neovascularization has been implicated as the most common cause of blindness and underlies the pathology of approximately twenty diseases of the eye. Both the growth and metastasis of solid tumors are also angiogenesis-dependent.

Although several angiogenesis inhibitors are currently available or under development for use in treating angiogenic diseases, there are disadvantages associated with these compounds. For example, suramin is a potent angiogenesis inhibitor that causes severe systemic toxicity in humans at doses required to reach antitumor activity. Other compounds, such as retinoids, interferons and antiestrogens appear safe for human use but have only a weak anti-angiogenic effect.

Thus, there is a need in the art for new compositions and methods for inhibiting angiogenesis associated with pathologic conditions.

### SUMMARY OF THE INVENTION

In one aspect, the invention provides a method of testing an agent for antiangiogenic activity. This method includes contacting an endothelial cell with the agent and assaying for activation of Rap-1 in the endothelial cell, activation of Rap-1 being indicative of antiangiogenic activity.

In another aspect, the invention provides a method of inhibiting angiogenesis in a cell population by contacting one or more of the cells in the population with an agent that activates Rap-1 or the Rap-1 signaling pathway.

In yet another aspect, the invention provides a method of treating a condition characterized by ocular neovascularization in a subject in need thereof by administering to the subject a therapeutically effective amount of an agent that activates Rap-1.

The invention also provides an antiangiogenic composition comprising an agent that activates Rap-1 or the Rap-1 signaling pathway, and a pharmaceutically acceptable excipient.

In a further aspect, the invention provides methods for activating the Rap-1 signaling pathway by expressing a polynucleotide encoding a constitutively active Rap-1 polypeptide that is operably connected to a promoter functional in a cell. Expression of the constitutively active Rap-1 polypeptide activates the Rap-1 signaling cascade and inhibits angiogenesis.

In another aspect, the invention provides kits for inhibiting angiogenesis comprising a polynucleotide encoding a constitutively active Rap-1 polypeptide.

In a still further aspect, the invention provides methods of inhibiting chemotaxis in a cell and inhibiting angiogenesis in a cell population by contacting one or more cells with an agent that inhibits Rho kinase.

### BRIEF DESCRIPTION OF THE FIGURES

FIG. 1A depicts cAMP concentration in HMVECs either left untreated or treated for 1 hour or 24 hours with Anthrax edema toxin (ET).

FIG. 1B shows phase contrast microscope images (100×) of HMVECs either left untreated or treated with ET for 24 hours.

FIG. 1C shows fluorescence microscopy images (200×) of actin stress fibers stained with Alexa Fluor 594 phalloidin in HMVECs either left untreated or treated with ET for 24 hours.

FIG. 2A depicts a graph showing results of a cell proliferation assay in HMVECs either treated with PA or ET or untreated for the indicated times.

FIG. 2B depicts a graph showing results of a cell migration assay in HMVECs treated with ET and plated in a modified Boyden chamber in the presence or absence of VEGF.

FIG. 2C shows fluorescence microscopy images of HMVECs either left untreated or treated with ET for 3.5 hours during formation of 3D tubes on MATRIGEL.

FIG. 2D shows phase contrast microscope images of HMVECs either left untreated or treated with ET at the indicated concentrations for 23 hours during formation of 3D tubes on MATRIGEL.

FIG. 3A depicts a graph showing microarray analysis of total RNAs in HMVECs either left untreated or treated with VEGF or VEGF+ET.

FIG. 3B depicts a graph showing real-time PCR analysis of transcript expression in HMVECs either left untreated or treated with VEGF or VEGF+ET.

FIG. 4A shows phase contrast microscope images (100×) of HMVECs either left untreated or treated with forskolin and IBMX or 8-(4-chlorophenylthio)-2′-O-methyladenosine-3′,5′cyclic monophosophate (“8CPT-2Me-cAMP”) at the indicated concentrations.

FIG. 4B depicts a graph showing chemotaxis inhibition in HMVECs treated with the indicated amount of either 6-Bnz-cAMP or 8CPT-2Me-cAMP in the presence or absence of VEGF in modified Boyden chambers.

FIG. 5A shows results of immunoblotting for activated Rap1 isolated from HMVECs either left untreated or treated with 8CPT-2Me-cAMP or ET for the indicated times. α-tubulin was immunoblotted to normalize for protein loading.

FIG. 5B shows results of immunoblotting for phosoCREB in lysates of HMVECs either left untreated or treated with forskolin+IBMX or 8CPT-2Me-cAMP for the indicated times.

FIG. 6A shows representative fields from microscopy images of immunostained sections of biodegradable scaffolds implanted in SCID mice and injected with the indicated concentrations of 8CPT-2Me-cAMP for 5 days.

FIG. 6B depicts a graph showing numbers of newly developed microvessels counted in 10 random fields of immunostained sections of biodegradable scaffolds implanted in SCID mice and injected with the indicated concentrations of 8CPT-2Me-cAMP for 5 days.

FIG. 7A is a photograph of an immunoblot using anti-HA to demonstrate expression of HA-tagged Rap1A63E and RapGAP in HMVECs.

FIG. 7B is a micrograph demonstrating expression of Rap1A63E caused the cells to flatten while expression of RapGAP caused cells to round.

FIG. 7C is a graph showing the constitutively active Rap-1 blocks VEGF-induced chemotaxis.

FIG. 7D is a graph showing that addition of a Rho kinase inhibitor blocks VEGF-induced chemotaxis.

FIG. 8 is a graph comparing the effect of expression of constitutively active Rap-1 to treatment with a cAMP analog on microvessel density as measured by counting CD31 positive microvessels.

FIG. 9 is a graph showing dose-dependent inhibition of in vivo FGF-induced angiogenesis in mouse corneal cells by treatment with a cAMP analog.

FIG. 10 is a set of photographs demonstrating viral delivery and expression of a polynucleotide to the mouse cornea.

### DETAILED DESCRIPTION OF SEVERAL EMBODIMENTS

The inventors have discovered that activation of the Epac/Rap-1 signaling pathway results in antiangiogenic effects in endothelial cells. The inventors have also discovered a number of effects of Rap-1 activation in endothelial cells that can be used to identify agents having antiangiogenic activity.

Cyclic adenosine monophosphate (cAMP) is a well-characterized second messenger that plays a role in a wide variety of cellular processes. Classically, the intracellular effects of cAMP were assumed to be mediated by protein kinase A (PKA). Recently, a family of Rap-1 guanine nucleotide exchange factors that are also directly activated by cAMP has been identified. These proteins, called Epacs (including Epac1 and Epac2), contain a cAMP binding pocket similar to the regulatory subunits of PKA. Activation of Rap-1 via Epac leads to a variety of cell signaling cascades that modulate integrins associated with the actin cytoskeleton, promote cell junction formation by cadherins, and regulate actin dynamics through mediators such as Rac, cdc42 and Rho.

A number of compounds have been identified that are capable of differentially activating the Epac/Rap-1 pathway, e.g., those described in WO 03/104250, which is incorporated herein by reference in its entirety. These compounds have been found to specifically bind to and modulate Epacs, but not PKA, both in vitro and in vivo. These compounds are taught to be useful in discriminating between Epac- and PKA-mediated pathways.

The invention provides methods for testing an agent for antiangiogenic activity by contacting an endothelial cell with the agent and assaying for activation of Rap-1 in the endothelial cell. Activation of Rap-1 is indicative of antiangiogenic activity. With reference to this and other embodiments of the invention, “contacting an endothelial cell with an agent” may occur directly or indirectly, and may occur in vitro, in vivo or ex vivo. The endothelial cell may be any type of endothelial cell and may be derived from any species. Examples of endothelial cells suitable for use in the methods of the invention include human umbilical vascular endothelial cells (HUVECs) and human microvascular endothelial cells (HMVECs). However, endothelial cells derived from any tissue may be used.

Suitably, the agent is provided in solution to a cell culture containing the endothelial cell. The agent may be continuously present after initial addition of the agent to endothelial cells or may be added for a period of time and then removed. The cell may be in contact with the agent at least about 15 minutes, at least about 1 hour, or at least about 24 hours, prior to assaying for activation of Rap-1. As will be appreciated, the agent suitably may be provided in a range of concentrations, e.g., by dilution.

One suitable assay useful in identifying Rap-1 activation is known as a “Rap-1 pull down assay,” described by Cullere et al., Blood 105(5) 1950-55 (2005) at page 1951, which is incorporated herein by reference. Briefly, cells are lysed in a suitable lysis buffer and clarified by centrifugation. Clarified lysates are incubated with a GST fusion protein containing the Rap-1 binding domain of Ral-GDS coupled to glutathione-sepharose beads. Proteins bound to the beads are then extracted and resolved by sodium dodecyl sulfate-polyacrylamide gel electrophoresis (SDS-PAGE) and subjected to western blot analysis using anti-Rap-1 antibodies.

Rap-1 activation may also be detected by assaying levels of mRNA encoding Epac-1, Epac-2 and/or RapGEF5, which are guanine nucleotide exchange factors (GEFs) for Rap-1. Analysis of expressed transcripts is suitably accomplished using standard methods, including oligonucleotide arrays, real-time PCR, and/or Northern blot analysis. The inventors have found that increased expression of any of these transcripts in endothelial cells treated with a Rap-1 activator relative to expression in control cells is indicative of antiangiogenic activity. Suitably, Epac-1, Epac-2 and/or RapGEF5 transcripts are induced at least about three-fold in cells treated with antiangiogenic agents. More suitably, transcripts are induced at least about seven-fold in treated cells.

The inventors have discovered that activation of Rap-1 via Epac in endothelial cells is indicative of antiangiogenic activity. An agent having antiangiogenic activity may prevent, inhibit and/or reverse neovascularization when administered to an in vitro, ex vivo or in vivo cell population. The cell population may be an endothelial cell population, or may be a tissue, organ or tumor cell population that includes multiple cell types. Thus, the presently described methods may be used to identify agents that are antiangiogenic via a Rap-1-mediated pathway. Rap-1 activation in an endothelial cell may be used alone or in combination with other assays to evaluate the antiangiogenic activity of an agent.

As described in the Examples below, cell flattening of endothelial cells contacted with an agent is indicative of the agent's antiangiogenic activity. Thus, evaluating an agent for the ability to cause endothelial cell flattening may be used in combination with an assay of Rap-1 activation.

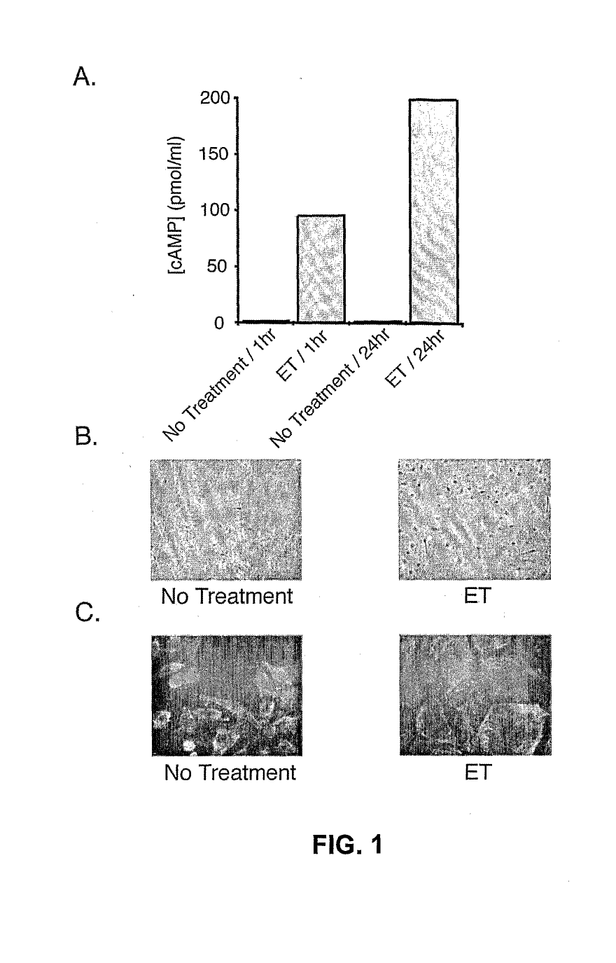

Endothelial cells in culture typically exhibit a rounded morphology. Contact with an antiangiogenic agent may result in a significant flattening effect. See, e.g., FIGS. 1B and 1C. Accordingly, “detecting cell flattening” refers to observing the morphology of endothelial cells subsequent to contacting the cells with the antiangiogenic agent and is relative to untreated or control treated cells. Cell flattening may be detected by any method known to those of skill in the art, e.g., by microscopic visualization of cultured cells. Alternatively, or in addition, cytoskeletal actin may be stained with a fluorophore- or chromaphore-labeled cytoskeletal marker, e.g., Alexa-phalloidin, rhodamine-phalloidin or bodipy-phallicidin, to facilitate visualization of cell flattening.

Evaluating putative antiangiogenic agents for the ability to cause endothelial cell flattening may be performed in a high throughput format as a screen prior to verifying antiangiogenic activity using a further assay. Accordingly, the present methods are suitable for screening large numbers of potential antiangiogenic agents. It is envisioned that several agents may be pooled and added to an endothelial cell culture. If cell flattening is detected in the culture after contact with a pool of agents, individual agents in the pool may be subjected to a further round of contacting an endothelial cell and detecting cell flattening, or agents causing cell flattening may be deduced by overlapping combinations of agents in the pools tested.

Endothelial cell migration toward chemotactic factors is another key feature of angiogenesis. As used herein, a “chemotactic factor” is a substance that directs cell locomotion (e.g., movement) in a concentration gradient of soluble extracellular agents. The term “chemotactic factor” may be used interchangeably with the term “chemoattractant.”

In the presently described methods, the ability of an agent to inhibit chemotaxis provides a further measure of antiangiogenic activity. Thus, a further step in some embodiments of the invention may include contacting an endothelial cell population with a putative antiangiogenic agent and evaluating chemotactic factor-induced chemotaxis in the endothelial cell population. Inhibition of chemotaxis is indicative of antiangiogenic activity.

“Inhibition,” used in the context of inhibiting chemotaxis, refers to either prevention or delay of chemotaxis by agent-treated cells vs. untreated or control treated cells. Suitably, chemotaxis of agent-treated cells is inhibited by at least about 5% relative to untreated or control treated cells. More suitably, chemotaxis of agent-treated cells is inhibited by at least about 50% in comparison to untreated or control treated cells. Most suitably, chemotaxis of agent-treated cells is inhibited by at least about 95% in comparison to untreated or control treated cells.

A variety of methods have been used to measure chemotaxis, including migration of cells under layers of agarose, phagokinetic tract analysis, cell orientation assays and time-lapse cinematography. Any of these methods may be used to screen agents for antiangiogenic activity according to the methods of the present invention. The chemotactic behavior of cells can also be assayed in a commercially available Boyden chamber, in which the cells are separated from a test substance by a membrane. Chemoattractants induce the migration of cells through the membrane into the compartment containing the chemotactic agent. Agarose gel clots are similarly used to measure migration or chemotaxis of cells in the presence of chemotactic compounds or to measure inhibition of migration by inhibitors of chemotaxis.

Vascular endothelial growth factor (VEGF) is a factor known to play a role in endothelial cell chemotaxis and is a common target of antiangiogenic therapies. Suitable methods of evaluating VEGF-induced endothelial cell chemotaxis are known in the art (e.g., Lingen M W, Methods Mol. Med. 78: 337-47 (2003), incorporated herein by reference). As will be appreciated, however, VEGF is but one chemotactic factor that can be utilized in the present methods. Other endothelial cell chemoattractants may also be used. TWEAK, a member of the tumor necrosis factor superfamily, binds to the Fn14 receptor of endothelial cells and is known to stimulate angiogenesis in vivo. Other suitable chemotactic factors include, but are not limited to, scatter factor/hepatocyte growth factor (SF/HGF), transforming growth factor (TGF)-alpha, TGF-beta1, TGF-beta2, epidermal growth factor (EGF), fibroblast growth factor (FGF)-1, FGF-2, insulin-like growth factor (IGF)-1, IGF-2, platelet-derived growth factor (PDGF)-M, PDGF-BB, pleiotrophin (PTN), and midkine (MK).

Following stimulation by pro-angiogenic factors, endothelial cells undergo dramatic morphological changes, including sprouting, migration, and differentiation into hollow tubes. Accordingly, a further step in some embodiments of the invention includes evaluating tubule formation in an endothelial cell population that has been contacted with a putative antiangiogenic agent. Evaluation of tubule formation can be evaluated in vitro using MATRIGEL impregnated with VEGF, as described by Kubota Y, et al., J. Cell Biol. 107:1589-98 (1988), incorporated herein by reference. Tube formation is suitably monitored by differential interference contrast and/or fluorescence microscopy. Treatment with agents exhibiting antiangiogenic activity will cause inhibition of the formation of tubules.

“Inhibition,” used in the context of tubule formation, refers to either prevention or delay of tubule formation by agent-treated cells vs. untreated or control treated cells. Suitably, tubule formation by agent-treated cells is inhibited by at least about 5% in comparison to untreated or control treated cells. More suitably, tubule formation by agent-treated cells is inhibited by at least about 50% in comparison to untreated or control treated cells. Most suitably, tubule formation by agent-treated cells is inhibited by at least about 95% in comparison to untreated or control treated cells.

A further assay for evaluating inhibition of tubule formation suitable for use in the presently described methods is an in vivo assay of microvessel density. In this regard, functional human microvessels can be engineered in SCID mice by implanting human dermal microvascular endothelial cells (HMVECs) seeded in biodegradable scaffolds, as described by Nor J E, et al., Lab Invest 81: 453-63 (2001), incorporated herein by reference. These cells organize into microvessels that anastomose with the host vasculature and transport mouse blood cells. Differences in vessel density are quantified post-euthanization by standard immunohistochemical techniques using, for example, anti-human CD31, and counting the number of CD31-positive vessels in a set number of random fields.

As it is known that Epacs are activated by cAMP, it is contemplated that one class of putative antiangiogenic agents that may be identified by the presently described methods are cAMP analogs. A cAMP analog is a molecule that is structurally and functionally related to cAMP. Several such cAMP analogs are described in International Application Publication No. WO 03/104250, which is incorporated herein by reference in its entirety. Other agents that may be identified as antiangiogenic in the present methods include adenylyl cyclases, which raise intracellular levels of cAMP.

Some embodiments of the invention provide a method of inhibiting angiogenesis in a cell population by contacting one or more cells in the population with an agent that activates Rap-1. Specifically, it is contemplated that methods of inhibiting angiogenesis will be useful in both in vitro and in vivo applications, including methods of treating angiogenesis-related conditions. Inhibiting angiogenesis includes blocking, reversing, limiting, or preventing angiogenesis, and suitably includes reducing microvessel density. Accordingly, the cell population contacted with the Rap-1 activating agent may be an in vitro, ex vivo or in vivo cell population. The cell population may be an endothelial cell population, or may be a tissue, organ or tumor cell population that includes multiple cell types. Suitably, at least two of the cells in the population are endothelial cells.

In some embodiments, angiogenesis may be inhibited in vitro to characterize the antiangiogenic activity of particular agents, or for other research-related purposes. In such in vitro methods, the cell population may include an excised tumor or other tissue, cultured primary cells or an established cell line. Contacting the cell population with the Rap-1 activating agent suitably may be accomplished directly or indirectly, e.g., by adding the agent to the media in which the cells are cultured or maintained.

One suitable agent for in vitro inhibition of angiogenesis is Anthrax edema toxin (ET). ET is a highly active calmodulin-dependent adenylyl cyclase that raises intracellular cAMP levels and causes a broad range of tissue damage upon host cell entry. The Examples demonstrate that ET activates Rap-1 in endothelial cells. Moreover, the inventors have surprisingly discovered that ET induces cytoskeletal changes in endothelial cells and exhibits antiangiogenic effects in in vitro and models of angiogenesis. In its native form, ET administered subcutaneously results in a local edema around the site of injection, even at very low doses. Thus, it is contemplated that ET may be suitable for inhibition of angiogenesis in vivo if it is modified to reduce or eliminate its toxic effects. In addition, other adenylyl cyclases may be useful in the methods of the present invention.

Other suitable agents for inhibition of angiogenesis include cAMP analogs. In the Examples, 8CPT-2Me-cAMP was shown to have antiangiogenic effects both in vitro and in vivo. It is contemplated that additional analogs of cAMP that activate Rap-1 will also function to inhibit angiogenesis.

In vivo inhibition of angiogenesis with an agent that activates Rap-1 in accordance with the present embodiments may be useful both for research and treatment purposes. In such in vivo methods, the Rap-1 activating agent may be administered by any method known to be suitable to the skilled artisan for delivering an agent to a particular site within the body. Suitably, the agent may be administered to a cell population intratumorally, e.g., to the tumor vasculature, or may be administered to ocular tissue, e.g., to retinal or subretinal structures. When administered to a tumor cell population, it is understood that the agent may be delivered systemically, regionally, or directly to a solid tumor. When administered to ocular tissue, the agent is suitably topically, intravitrally or periocularly administered. Determination of a suitable route of administration and dosing regimen is within the skill in the art.

Although the process of angiogenesis is not completely understood, a number of pathological conditions have been definitively associated with abnormal or harmful angiogenesis. Accordingly, methods of treating these conditions in a subject in need thereof are provided by the present invention. Treatment may be accomplished by administering a therapeutically effective amount of an agent that activates Rap-1 in endothelial cells to a subject in need thereof. Suitably, a therapeutically effective amount is an amount that has sufficient antiangiogenic activity to afford at least some therapeutic benefit. Such therapeutically effective antiangiogenic activity suitably inhibits endothelial cell migration and tubule formation in particular tissues relative to suitable control tissue.

Angiogenesis has been associated with a number of different types of cancer, including solid tumors of the breast, eye, prostate, brain, pancreas, lung, stomach, ovary and cervix, as well as blood-borne tumors. Examples of particular cancers include rhabdomyosarcomas, retinoblastoma, Ewing's sarcoma, neuroblastoma, and osteosarcoma. Angiogenesis is a prerequisite to solid tumor growth and thus inhibition of angiogenesis is one way to limit solid tumor growth. Angiogenesis is also associated with blood-borne tumors, such as leukemias, any of various acute or chronic neoplastic diseases of the bone marrow in which unrestrained proliferation of white blood cells occurs, usually accompanied by anemia, impaired blood clotting, and enlargement of the lymph nodes, liver and spleen. It is believed that angiogenesis plays a role in these abnormalities in the bone marrow that give rise to leukemia tumors and multiple myeloma diseases.

Further conditions mediated by angiogenesis are those characterized by ocular neovascularization. This constellation of diseases is characterized by invasion of new blood vessels into the structures of the eye, such as the retina or cornea. Ocular neovascularization is the most common cause of blindness and is involved in approximately twenty eye diseases. In age-related macular degeneration, the associated visual problems are caused by an ingrowth of choroidal capillaries through defects in Bruch's membrane with proliferation of fibrovascular tissue beneath the retinal pigment epithelium. Angiogenic damage is also associated with diabetic retinopathy, retinopathy of prematurity, corneal graft rejection, neovascular glaucoma, and retrolental fibroplasia. Other diseases associated with corneal neovascularization include, but are not limited to, epidemic keratoconjunctivitis, Vitamin A deficiency, contact lens overwear, atopic keratitis, superior limbic keratitis, rubeotic glaucoma, interstitial keratitis and pterygium keratitis sicca.

In the context of the invention, “treating” or “treatment” of a cancer or tumor in a mammal includes one or more of: (1) inhibiting growth of the cancer, i.e., arresting its development; (2) preventing spread of the cancer, i.e. preventing metastases; (3) relieving the cancer, i.e., causing regression of the cancer, (4) preventing recurrence of the cancer; (5) palliating symptoms of the cancer; (6) promoting rejection of one or more solid tumors; and (7) reducing tumor volume.

“Treating” or “treatment” of ocular neovascularization-associated conditions includes, but is not limited to, restoring vision or preventing, reducing or mitigating vision loss or reducing neovascularization (e.g., microvessel density) of the eye.

Administration of a Rap-1 activating agent to a subject in accordance with the present invention appears to exhibit beneficial effects in a dose-dependent manner. Thus, within broad limits, administration of larger quantities of Rap-1 activating agent is expected to exhibit efficacy to a greater degree than does administration of a smaller amount. Moreover, efficacy is also contemplated at dosages below the level at which toxicity is seen. Further, in practice, higher doses are generally used where the therapeutic treatment of a disease state is the desired end, while the lower doses are generally used for prophylactic purposes.

It will be appreciated that the specific dosage administered in any given case will be adjusted in accordance with the specific compounds being administered, the disease to be treated, the condition of the subject, and other relevant medical factors that may modify the activity of the agent or the response of the subject, as is well known by those skilled in the art. For example, the specific dose for a particular patient depends on age, body weight, general state of health, on diet, on the timing and mode of administration, on the rate of excretion, and on medicaments used in combination and the severity of the particular disorder to which the therapy is applied. Dosages for a given patient can be determined using conventional considerations, e.g., by customary comparison of the differential activities of the Rap-1 activating agent and of a known agent, such as by means of an appropriate conventional pharmacological protocol.

The maximal dosage for a subject is the highest dosage that does not cause undesirable or intolerable side effects. However, particularly with regard to treating such severe conditions as vision loss or cancer, a cost-benefit analysis may allow for administration of dosages which would otherwise not be used. The number of variables in regard to an individual treatment regimen is large, and a considerable range of doses is expected. It is anticipated that dosages of Rap-1 activating agent in accordance with the present invention will reduce symptoms at least 50% compared to pre-treatment symptoms.

The present invention also provides pharmaceutical compositions or preparation comprising a Rap-1 activator, or a physiologically/pharmaceutically acceptable salt thereof, and may further comprise a physiologically/pharmaceutically acceptable carrier and/or excipient to facilitate administration of the Rap-1 activator to a patient. The composition or preparation may be a solid, semisolid or liquid preparation (tablet, pellet, troche, capsule, suppository, cream, ointment, aerosol, powder, liquid, emulsion, suspension, syrup, injection, etc.) suitable for selected mode of administrating the Rap-1 activator in a pharmaceutically acceptable excipient. As indicated above, adenylyl cyclases and cAMP analogs are known activators of Rap-1 and may be used to provide antiangiogenic compositions. Other activators of Rap-1 may be identified using the methods described herein.

The present invention also provides methods for inhibiting angiogenesis in a cell population by expressing in one or more cells a polynucleotide encoding a constitutively active Rap-1. In the Examples, the constitutively active Rap-1 mutant Rap-1 A63E was found to have antiangiogenic activity in HMVECs expressing Rap-1 A63E. It is envisioned that any constitutively active Rap-1 could be use, including, for example, Rap-1 G12V. In order to obtain expression, the polynucleotide was operably connected to a promoter functional in the transformed cell. The polynucleotides can be expressed by endothelial cells or by other cells in the region of the endothelial cells such that the expressed polypeptides inhibits angiogenesis in the cell population. Cells suitable for use include primary cells, cultured cells and cells derived from embryonic or other stem cells. The cell can be located in a subject, suitably a mammalian subject, or it can be in vitro.

The polynucleotide can be introduced into the cell by any suitable means, including electroporation, transformation, transfection, liposome delivery or any other means known in the art. The polynucleotide may be introduced into a cell in vitro, in vivo or ex vivo. If the polynucleotide is introduced into the cell in vitro or ex vivo, the cell may be implanted in vivo after the polynucleotide is introduced into the cell. In one embodiment, a vector comprises the polynucleotide and delivers the polynucleotide to the cell. Vectors include, but are not limited to liposome and viral vectors such as an adenoviral vector, an adeno-associated viral vector, a vaccinia viral vector, a retroviral vector, a pox viral vector and a herpesviral vector. In the Examples, a lentivirus vector was used.

The present invention also provides methods of inhibiting chemotaxis in a cell by contacting one or more cells with an agent that inhibits Rho. In addition, methods are provided for inhibiting angiogenesis in a cell population by contacting one or more cells with an agent that inhibits Rho. The Examples demonstrate that an inhibitor of Rho kinase (ROCK) inhibits chemotaxis. Inhibition of Rho by activated Rap-1 suppresses chemotaxis. Thus it is contemplated that direct inhibition of Rho via addition of agents that inhibit Rho effector function may also inhibit angiogenesis. Agents that inhibit Rho may be used in vitro, in vivo or ex vivo. The agents may be administered intratumorally, topically or by any other means known in the art. Agents that inhibit Rho may be used to treat conditions characterized by neovascularization such as tumors and ocular diseases in ways similar to the activators of Rap-1 described above.

The following examples are provided to assist in a further understanding of the invention. The particular materials and conditions employed are intended to be further illustrative of the invention and are not limiting upon the reasonable scope of the appended claims.

### EXAMPLES

### Example 1

### Experimental Methods

The following materials and methods were used in the experiments described in Examples 2-12.

Materials—Protective antigen (PA) was purchased from List Biological Laboratories (Campbell, Calif.). Alexa Fluor 594 phalloidin was purchased from Molecular Probes (Eugene, Oreg.). 8CPT-2Me-cAMP and PKA activator N⁶-Benzoyladenosine-3′,5′cyclic monophosphate (6-Bnz-cAMP) were from Biolog Life Science Institute (Bremen, Germany). Rabbit polyclonal antibody against Phospho-CREB (Serl33) was from Cell Signaling Technology (Beverly, Mass.). Rabbit polyclonal antibody against Rap1 and a mouse monoclonal antibody against α-tubulin were from Santa Cruz Biotechnology (San Diego, Calif.). Mouse anti-human CD31 was from DAKO (Carpinteria, Calif.). Forskolin, IBMX and recombinant human endostatin were from Sigma-Aldrich (Saint Louis, Mo.). Di-TSPa was from Abbott Laboratories (Abbott Park, Ill.). Recombinant human vascular endothelial growth factor (rhuVEGF) was from Alpha Diagnostics International (San Antonio, Tex.), and MATRIGEL was purchased from Becton Dickinson and Company (Franklin Lakes, N.J.). EF was produced in E. coli using standard recombinant technology, e.g., as described in Soelaimain S, et al., J. Biol. Chem. 278: 25990-7 (2003), which is incorporated herein by reference.

Cell culture—Human dermal microvascular endothelial cells (HMVECs) (Cell Systems, Kirkland, Wash.) were cultured using standard culture methods, e.g., as described in Cline E I et al., Cancer Res. 62: 7143-48 (2002), incorporated herein by reference and Khodarev N N et al., J. Cell Sci. 116: 1013-22 (2003), incorporated herein by reference. For the tubule formation assay, neonatal dermis human microvascular endothelial cells (HMVECs) were obtained at passage 3 from Cascade Biologics, Inc. (Portland, Oreg.) and routinely subcultured as recommended by the manufacturer. For treatment with ET, cells were pre-treated with PA for 15 minutes before adding E. coli-produced EF.

Assay of cellular cAMP—HMVECs were seeded at 6×10⁴ cells per plate in 35 mm plates. Two days later, cells were treated with or without ET for indicated times and lysed with 300 μl of 0.1 N HCl. 50 μl of lysates were applied for measurement of cAMP using Assay Designs' Direct cAMP assay kit, nonacetylated version (Ann Arbor, Mich.).

Actin stress fiber staining—HMVECs were seeded directly on 35 mm plates at 30% confluence one day before treatment. Cells were then treated for 24 hours and actin stress fibers were stained with Alexa Fluor 594 phalloidin (3 U/ml) as described in, e.g., Li L et al., Mol. Cell Biol. 22: 1203-14 (2002), incorporated herein by reference.

Cell proliferation assay—HMVECs were seeded at 2000 cells per well in 96-well plates one day before treatment. Cells were then treated with or without ET for the indicated times. Cell proliferation was quantified using Promega's CellTiter 96 Aqueous One Solution Cell Proliferation Assay kit (Madison, Wis.).

Endothelial Cell Migration Assay—The endothelial cell migration assay was performed as described in, e.g., Lingen M W, Methods Mol Med. 78: 337-47 (2003), incorporated herein by reference. Briefly, HMVECs were starved overnight in media containing 0.1% bovine serum albumin (BSA), harvested, resuspended in DME with 0.1% BSA, plated on the bottom side of a modified Boyden chamber (Nucleopore Corporation, MD), and allowed to attach in the inverted chamber for 2 hours at 37° C. The chamber was then re-inverted, test substances added to the wells of the upper chamber and cells were allowed to migrate for four hours at 37° C. Membranes were recovered, fixed and stained and the number of cells that had migrated to the upper chamber per ten high power fields was counted. Background migration in DME+0.1% BSA was subtracted and the data reported as the number of cells migrated per 10 high power fields (400×). VEGF (R&D Systems, Minneapolis, Minn.) was used as a positive control at a concentration of 100 pg/ml.

Formation of 3D-Tubes on MATRIGEL—Freshly thawed MATRIGEL solution (SI) was combined 1:1 with Medium 131 and supplemented with 50 ng/mL rhuVEGF. Approximately 2.5 to 3.0 mL of this mixture was applied to pre-chilled 100 mm plastic tissue culture plates and incubated overnight at 37° C. in a humidified CO₂ incubator. For tube formation, subconfluent HMVECs at passage 5 to 6 were plated onto the MATRIGEL-coated dishes with addition of purified rhuVEGF at 50 ng/mL to the media at 2.25×10⁴ cells/cm². Tube formation was monitored via light, Nomarski differential interference contrast (DIC) and fluorescence microscopy, the latter using Calcein AM as an intracellular stain (Molecular Probes).

Microarrays—Subconfluent HMVECs (1×10⁶ in T25 flask) were starved overnight in EBM2 medium containing 0.1% BSA and treated with various reagents as indicated. Total RNA were extracted using Qiagen's RNeasy Mini kit (Valencia, Calif.). Hybridization was performed at the Functional Genomics Facility at the University of Chicago using Affymetrix's Human Genome U133 plus 2.0 Array. Data were analyzed with dCHip analyzer 1.3. Thresholds for selecting differentially expressed genes were set at relative difference≧2 fold and absolute difference≧100.

Relative quantification of Epac-1, Epac-2 and RapGEF5 mRNA expression using real-time PCR—Reverse transcription was carried out according to the 2-step method by RETROScript (Ambion). Briefly, 1 μg of mRNA was amplified with 2 μl of random decamer primers in a Gene Amp PCR System 9700 thermocycler (Applied Biosystems). ASSAYS-ON-DEMAND for Epac-l, Epac-2, RapGEF5 and GAPDH were purchased from Applied Biosystems and contained unlabeled forward and reverse PCR primers as well as a FAM dye-labeled probe. Reactions were performed in triplicate according to the manufacturer's recommendations. Samples contained 50 ng of cDNA in a total volume of 50 μl. Amplification and detection of cDNA fragments was performed in a sealed 96-well microtiter plate in an ABI Prism 7300 spectrofluorometric thermocycler (Applied Biosystems) using the following conditions: 2 minutes at 50° C. followed by 10 minutes at 95° C. then 40 cycles of 15 seconds at 95° C. and 1 minute at 60° C. Analysis was performed using the RQ Study Software Version 1.2.3 (Applied Biosystems).

Sub-Cloning of Rap1A63E and Rap1GAP. cDNAs encoding Rap1 or Rap1GAP (Castro et al., J Biol. Chem. 278:32493-32496 (2003)) were PCR amplified from pCGN-Rap1A63E and pFLAG-CMV2-RapGAP, respectively, using primers that encode an N-terminal hemagglutinin (HA)-tag and subcloned into pGEM-T-Easy (Promega, Madison, Wis.). HA-Rap1A63E and HA-Rap1GAP were then subcloned into the pCDH-MCS1-EF1-copGFP vector (Systems Biosciences, Mountain View, Calif.) using Not I restriction endonuclease (Invitrogen, Carlsbad, Calif.). DNA sequencing was performed at the University of Chicago Cancer Center DNA Sequencing Facility and revealed one synonymous mutation in Rap1 GAP (GGC→GGT corresponding to codon 326).

Lentiviral transduction of HMVECs. Production of lentivirus was performed by contransfecting pCMV-VSV-G and pCMVΔR8.2 (Naldini et al., PNAS 93:11382-11388 (1996)) into 293T cells along with the pCDH-GFP (empty vector control), pCDH-Rap1A63E-GFP, or pCDH-Rap1GAP-GFP using TransIT®-LTI (Mirus, Madison, Wis.). Viral supernatants were collected 48 hours after transfection and added to HMVECs in the presence of 8 μg/ml hexadimethrine bromide (Sigma, St. Louis, Mo.) for 2.5 hours at 37° C. The percentage of cells expressing GFP was assessed by fluorescence microscopy and flow cytometry.

Cell lysate extraction and western analysis—Cell lysates were collected, resolved by SDS-PAGE, and analyzed by western blot using anti-Phospho-CREB (Ser133) antibody or anti-tubulin antibody at a dilution of 1:1000 or anti-Rap1 at a dilution of 1:500, using standard methods as described in Kuo, W L et al., J. Biol. Chem. 279: 23073-81 (2004), incorporated herein by reference.

Rap1 pull down assay—HMVECs were grown to subconfluent in 100 mm plates, starved in EBM2 medium plus 0.1% BSA over night and treated as indicated. Rap1 pull down assays were performed using GST-RaIGDS-RBD beads as described in Cullere, X et al., Blood 105: 1950-5 (2005), incorporated herein by reference.

SCID Mouse Model of Human Angiogenesis—Functional human microvessels were induced in severe combined immunodeficient (SCID) mice (CB-17 SCID; Taconic, Germantown, N.Y., USA). Briefly, 1×10⁶ HMVECs were seeded in poly-(L-lactic acid) (PLLA, Medisorb, Cincinnati, Ohio, USA) biodegradable scaffolds, and 2 scaffolds were implanted subcutaneously into the dorsum of each SCID mouse. Beginning 5 days after implantation, the animals received a daily local injection of 0.03 or 0.3 μmoles 8CPT-2Me-cAMP or vehicle control (20 mM Tris HCl, pH 8.0 and 150 mM NaCl) in a volume of 50 μl/scaffold. The injections continued for 5 days, and mice were euthanized 24 hours after the last injection. The implants were retrieved, fixed in 10% neutral buffered formalin for 30 min at 4° C., and processed for histology. The care and treatment of mice were in accordance with the University of Chicago's Institutional Guidelines.

Determination of In Vivo Microvessel Densities—Formalin fixed, paraffin-embedded implants were generated in order to quantify differences in vessel densities in the tumors of the treated and untreated animals. Serial 5 μm sections were processed and deparaffinized for standard immunohistochemistry. Antigen epitopes were unmasked by microwaving the specimens in EDTA-Tris buffer (1 mM EDTA pH 9.0, 1 mM Tris pH 7.0). Non-specific blocking was achieved by using 5% milk peroxidase for 20 minutes. Tissue sections were incubated with a 1:100 dilution of primary antibody mouse anti-human CD31 (DAKO, Carpinteria, Calif.) for 30 minutes at 37° C. and the secondary antibody Envision+Mouse for 30 minutes at 37° C. Liquid DAB+ substrate chromagen system (DAKO, Carpinteria, Calif.) was applied to visualize the bound antibody. The number of CD31+blood vessels was counted in 10 random fields per sponge using an optical microscope (×400).

### Example 2

### Anthrax Edema Toxin Activates cAMP and Induces Cytoskeletal Changes in Vascular Endothelial Cells

As shown in FIG. 1A, treatment of human microvascular endothelial cells (HMVECs) with ET resulted in the rapid intracellular accumulation of cAMP that more than doubled by one day following treatment. As shown in FIG. 1B, examination of HMVEC cell morphology revealed a dramatic change in shape from the normal oval morphology to a flattened morphology. Similar results were observed upon treatment of human umbilical vein endothelial cells (HUVECs) with ET (data not shown). As shown in FIG. 1C, this change in morphology and the underlying actin cytoskeleton was more clearly illustrated by staining actin in ET-treated versus untreated HMVECs with Alexa-labeled phalloidin. Taken together, these results indicate that EF can induce significant morphological and cytoskeletal changes in vascular endothelial cells.

### Example 3

### Anthrax Edema Toxin Inhibits Chemotaxis and Tube Formation but not Cell Proliferation in Vascular Endothelial Cells

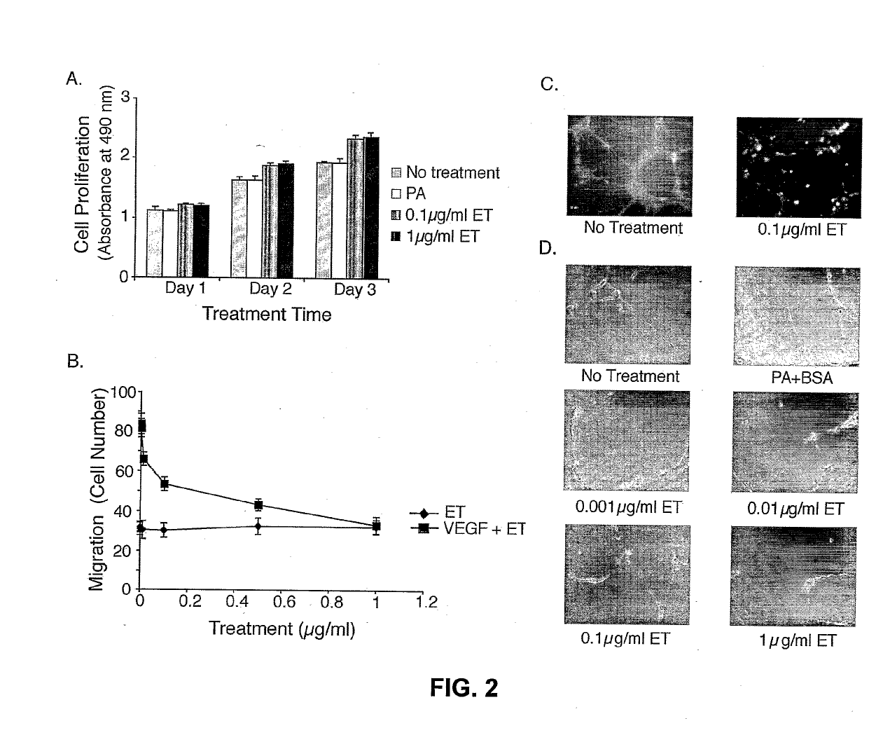

A cell proliferation assay was used to analyze the possibility of cell growth inhibition by cAMP. As shown in FIG. 2A, not only was there no suppression of cell growth, but ET actually appeared to enhance cellular proliferation at a low but statistically significant level. These results suggest that the changes in cell shape are unlinked to growth defects.

As an in vitro measure of chemotactic migration, HMVECs were placed in a Boyden chamber and the number of cells that migrate toward a VEGF stimulus were quantitated. As shown in FIG. 2B, when cells were pretreated with ET, the number of cells that underwent VEGF-induced migration decreased in a dose-dependent manner with an ID₅₀ of 0.07 μg/ml. All modes of migration were not inhibited by ET, as the fraction of cells migrating in the absence of VEGF was the same independent of ET treatment. Thus, ET inhibition was specific for the chemotactic response of the endothelial cells to VEGF, not background migration.

Next, it was determined whether ET has an inhibitory effect on tubule formation. ET caused a delay in the formation of the tubular network as evidenced by an alteration in temporal progression and final morphology. As shown in FIG. 2C, the appearance of the endothelial cells after addition of 0.1 μg/ml ET for 3.5 hours was characteristic of earlier stages of tube formation, as shown by fluorescent staining with the cell permeant Calcein AM that localizes in viable cells. Treatment of endothelial cells for up to one day showed a dose-dependent decrease in long range networks formed, accompanied by an increase in adhesed cell clustering at nodes and an almost complete absence of long, thin connecting cells (FIG. 2D). These results indicate that ET has an inhibitory effect on endothelial network formation, possibly as a result of inhibiting chemotaxis in cultured endothelial cells.

### Example 4

### Anthrax Edema Toxin Induces Expression of Rap GEFs

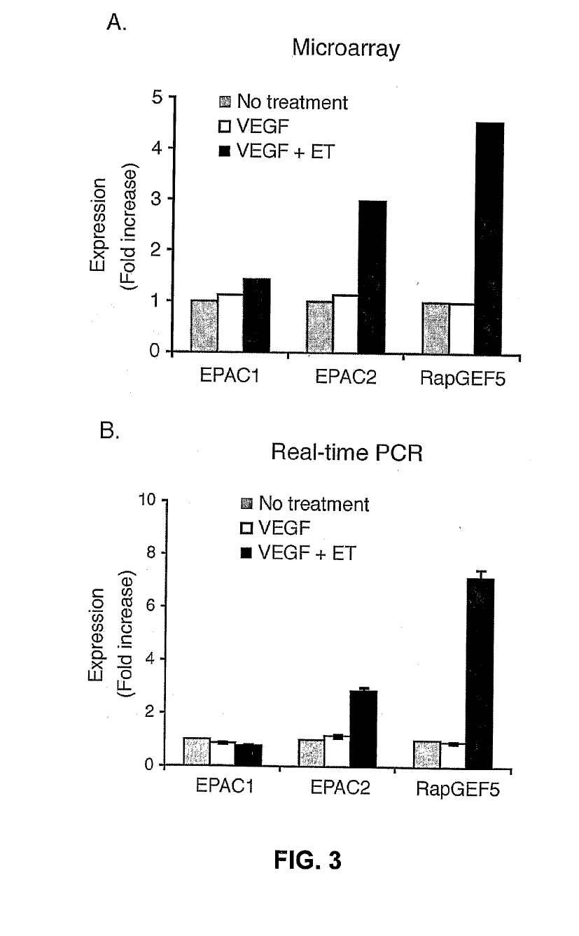

To determine what genes might be involved in mediating the ET effect on VEGF-activated endothelial cell migration, RNA was isolated from HMVECs that were either left untreated, stimulated with VEGF, or stimulated with VEGF in the presence of ET. Since the effects on chemotaxis could be observed by 4 hours of ET treatment, RNA from cells after treatment for the same 4 hour time period was analyzed. Analysis of the expressed transcripts using oligonucleotide arrays revealed a number of genes whose expression was altered by ET. Most of these changes involved suppression of genes known to be VEGF-induced (data not shown). However, there were a few examples of genes that were induced specifically by ET. As shown in FIG. 3A, expression of two genes which code for guanine nucleotide exchange factors (GEFs) for Rap1 were induced by ET treatment. The cAMP-dependent RapGEF4 (also called Epac2) and the cAMP-independent RapGEF5 transcripts were induced 3 to 7 fold by ET as assessed by real-time PCR. This induction was not a generalized effect, as no similar enhancement of RapGEF3 (Epac1) transcription was observed. Since RapGEFs activate Rap isoforms, these results suggest that the cAMP generated by the cells in response to ET not only activates some of the RapGEF proteins directly but also induces their expression in a positive feedback loop.

### Example 5

### Activated Epac Causes Morphological Changes and Inhibits Chemotaxis in Microvascular Endothelial Cells

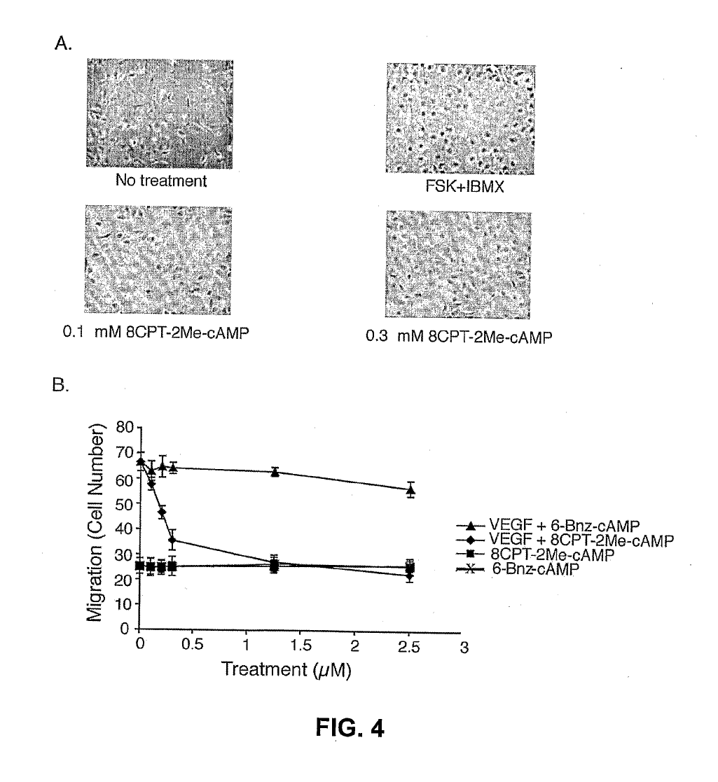

Since ET can both activate and induce GEFs for Rap1 such as Epac2, we investigated whether selective activation of Epac could mimic the effects on endothelial cells elicited by ET. To confirm that cAMP was the mediator of the ET effects, HMVECs were treated with forskolin plus a phosphodiesterase inhibitor (IBMX) to induce endogenous adenylyl cyclase activity. As observed in FIG. 4A, the endothelial cells also flattened in response to cAMP generation, and this effect could be mimicked by treatment of cells with 8CPT-2Me-cAMP.

Since endothelial cell migration toward chemotactic factors is a key feature of angiogenesis, it was determined whether Epac activation can similarly block chemotaxis. As shown in FIG. 4B, 8CPT-2Me-cAMP inhibited VEGF-stimulated HMVEC cell migration with an ID₅₀ of 0.2 μM and complete inhibition at 2.5 μM. Again, no suppression of nonspecific cell migration was observed. In contrast, treatment of HMVECs with 6-Benz-cAMP, a selective activator of Protein Kinase A, had no effect on endothelial cell migration. These results indicate that specific Epac activation can mimic ET-induced suppression of VEGF-induced chemotaxis.

### Example 6

### Rap1 is Activated by ET and 8CPT-2Me-cAMP

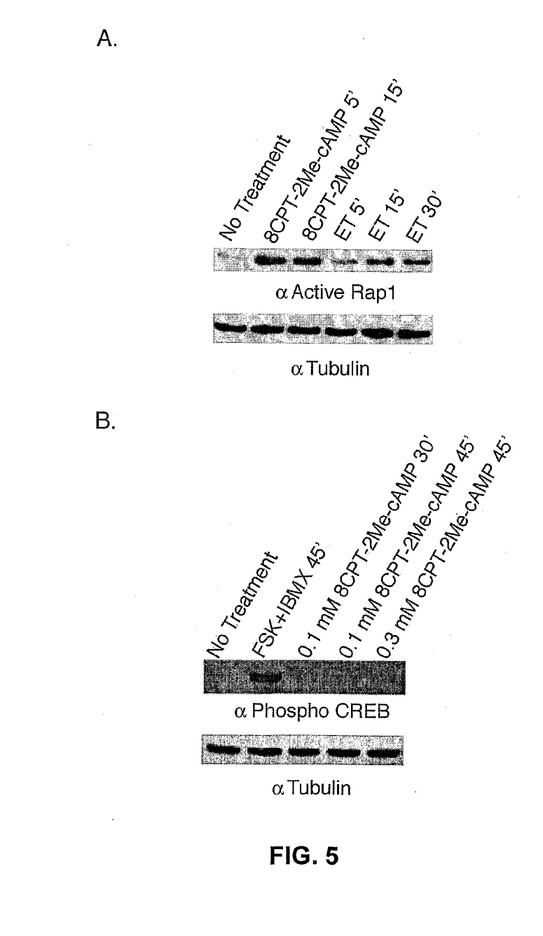

As shown in FIG. 5A, Epac activator 8CPT-2Me-cAMP is a robust stimulator of Rap1 as shown by the pull down assay for GTP-bound Rap1. Stimulation with ET at a concentration that blocks chemotaxis in HMVECs also induces Rap1 activation that is maintained for at least 30 minutes. To confirm that the Epac activator was not stimulating PKA, we analyzed CREB, a substrate of PKA, in treated cells. In contrast to forskolin, which robustly stimulated CREB phosphorylation, the 8CPT-2Me-cAMP activator of Epac did not induce significant CREB phosphorylation (FIG. 5B). These results suggest that Rap1 activation can account for the observed physiological effects of ET and 8CPT-2Me-cAMP on vascular endothelial cells and may contribute to the action of anti-angiogenic factors.

Some anti-angiogenic agents such as thrombospondin-1 promote endothelial cell death via apoptosis. However, in contrast to the thrombospondin-1 mimetic peptide Di-TSPa, we did not detect apoptotic induction by 0.3 mM 8CPT-2Me-cAMP in endothelial cells even in low serum conditions (data not shown). These results are consistent with previous results showing that ET did not inhibit cell proliferation. Thus, the effects of ET and Epac on vascular endothelial cells appear to be related to cytoskeletal-based processes such as cellular morphology and long range migration rather than proliferation and cell survival.

### Example 7

### Epac Activators Block in Vivo Angiogenesis in Mice

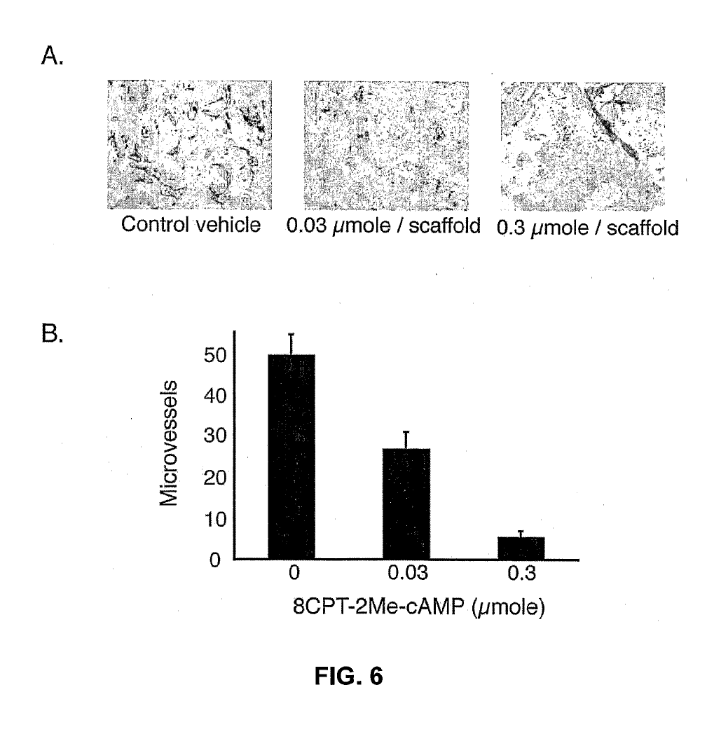

An in vivo model system was used to evaluate the effect of 8CPT -2Me-cAMP on microvessel development in SCID mouse scaffold implants populated with HMVECs. Each implant was injected with either 0.03 μmoles of 8CPT-2Me-cAMP or 0.3 μmoles of 8CPT-2Me-cAMP or was left untreated. Tissue slices were stained with anti-CD31 antibody and quantitated for microvessel density. As shown in FIG. 6B, the number of microvessels was inhibited by roughly 50% with 8CPT-2Me-cAMP at the lower dose and inhibited by greater than 90% at the higher dose. These results demonstrate that Rap1 activation via Epac is sufficient to suppress normal microvascular angiogenesis.

### Example 8

### Constitutively Activated Rap1 Blocks Chemotaxis

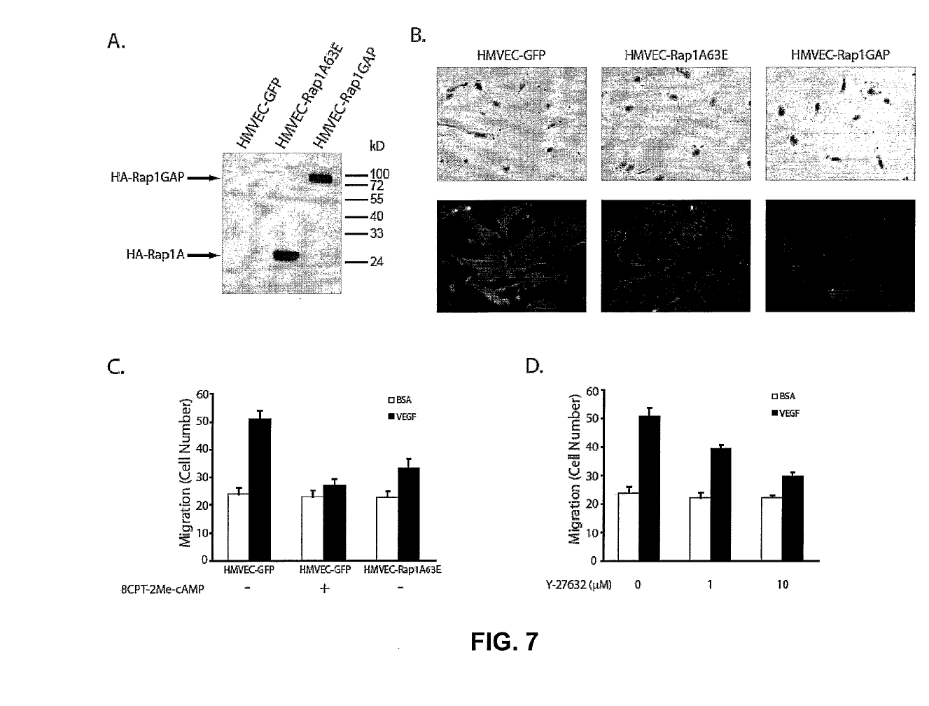

To test whether activated Rap1 is indeed sufficient to inhibit chemotaxis, HMVEC cells were transduced with a lentivirus expressing the activated Rap1A63E mutant as well as a GFP marker. Over 95% of the cells expressed GFP by three days, and the exogenous Rap1 was readily detected by immunoblotting (FIG. 7A). The Rap1-expressing cells also exhibited the flattened phenotype and actin changes observed upon 8CPT-2Me-cAMP treatment (FIG. 7B). Analysis of the mutant Rap1-expressing HMVECs in a chemotaxis assay either in the presence of absence of the Epac activator showed a potent suppression of VEGF-induced chemotaxis (FIG. 7C). To assess the requirement for Rap1 in 8CPT-2Me-cAMP action, cells were also transduced with lentivirus expressing Rap1GAP. Cells expressing Rap1GAP rounded up (FIG. 7B); however, robust Rap1GAP expression caused loss of matrix attachment, preventing further analysis. Taken together, these results demonstrate that Rap1 is sufficient to suppress VEGF-induced chemotaxis in HMVEC cells and implicates Rap1 in the morphological changes induced by Epac activators.

### Example 9

### Rho Kinase Inhibitors Block Chemotaxis

Rho has previously been shown to be inhibited by Epac activation following a thrombin stimulus in HMVECs (Cullere et al., Blood 105:1950-1955 (2005) and Fukuhara et al., Mol. Cell Bio 25:136-146 (2005)). Therefore, the role of the Rho signaling cascade in chemotaxis was investigated. Reproducible dose-dependent inhibition of VEGF-induced chemotaxis was observed after co-treatment of cells with an inhibitor (Y-27632) of the downstream Rho effector, Rho kinase (ROCK) (FIG. 7D). The data demonstrate a requirement for this pathway, although the inhibition was not complete suggesting that other factors also play a role. These results suggest that inhibition of Rho by activation of Rap1 is one mechanism by which Epac-activated Rap1 suppresses chemotaxis in HMVECs.

### Example 10

### Constitutively Activated Rap-1 Inhibits Angiogenesis in Mice

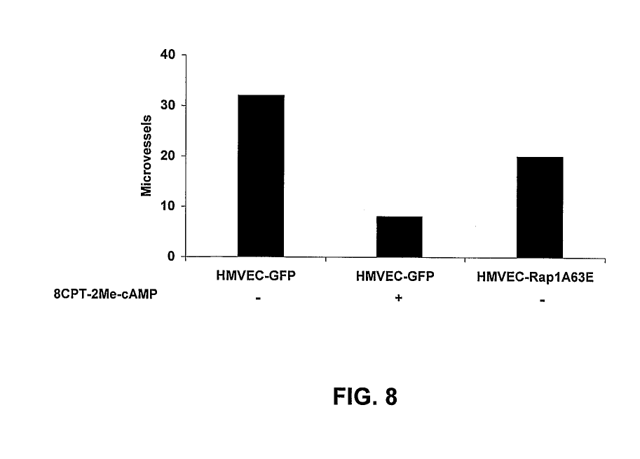

To confirm that activated Rap1 in HMVECs is sufficient to prevent angiogenesis in vivo, HMVECs expressing the constitutively active Rap1 (FIG. 8) were tested in biodegradable scaffolds. Attempts to access the effect of RapGAP in HMVECs were unsuccessful due to anoikis of the cells prior to implantation (see cell rounding in FIG. 7B). Following implantation, tissue slices were analyzed for CD31 expression. Significant inhibition of angiogenesis in HMVECs expressing Rap1 was detected (FIG. 8), although inhibition was less than that observed for HMVECs injected with the 8CPT-2Me-cAMP. These results indicate that Rap1 activation is sufficient to inhibit angiogenesis of human endothelial cells under physiological conditions.

### Example 11

### Inhibition of FGF-Induced Angiogenesis in Mouse Cornea

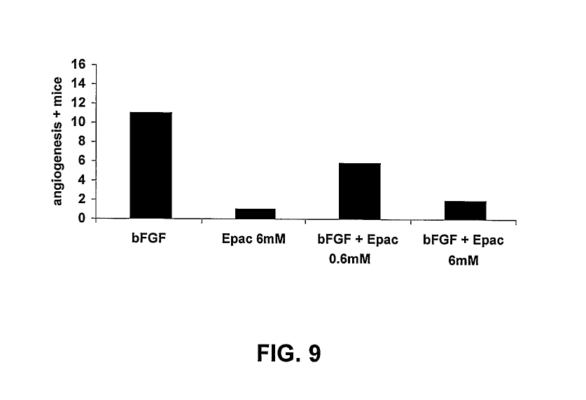

The effect of 8CPT-2Me-cAMP treatment on FGF-induced angiogenesis was investigated using the mouse corneal assay. Mice were injected with either 0.6 or 6 μmoles of 8CPT-2Me-cAMP, concentrations similar to those used for the skin implant scaffolds. The results, shown in FIG. 9, demonstrate that the higher dose of 8CPT-2Me-cAMP effectively suppressed FGF-induced angiogenesis in the mouse cornea. Only 1 out of 10 mice showed angiogenesis and that was due to corneal damage. At the lower dose of 8CPT-2Me-cAMP, 6 out of 8 mice had detectable vascularization (compared to 6 out of 6 in control mice), but the average area of vascularization was about half that of control mice. Thus, these results demonstrate that 8CPT-2Me-cAMP is a potent angiogenic inhibitor that functions in both skin and eye.

### Example 12

### Expression of GFP in Mouse Cornea

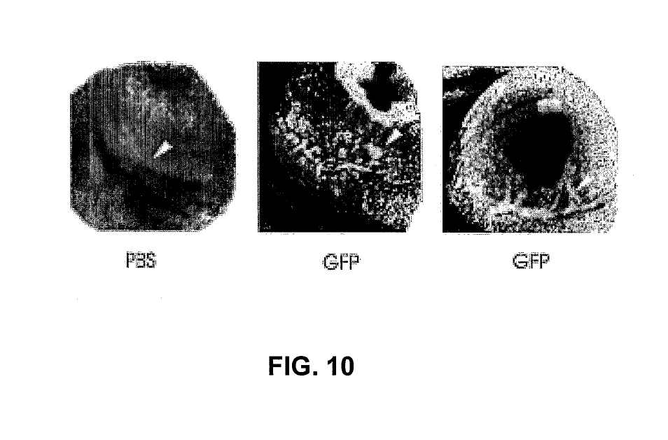

A mouse corneal model using lentivirus expressing GFP from a CMV promoter was developed to evaluate its potential utility in ultimately expressing Rap1 in mouse cornea to test its role as an inhibitor of different angiogenic stimuli or as a mediator of anti-angiogenic agents in vivo. The lentivirus (appr. 1×10⁶/ml) expressing GFP from a CMV promoter was injected into the tail vein of mice and the cornea was monitored. GFP expression served as a marker to determine efficiency of delivery. As shown in FIG. 10, we were able to deliver GFP-tagged lentivirus into mouse corneas. A bFGF pellet was present in all three mice. The vessels are indicated by arrows in FIG. 10, and can be readily detected by fluorescence in cornea injected with GFP virus. Note the lack of fluorescence where PBS rather than virus was injected. These results establish the feasibility of the viral injection approach as a powerful in vivo assay.

### Example 13

### Treatment of Macular Degeneration with Rap-1 Activating Compounds

A patient complaining of vision loss is diagnosed with age-related macular degeneration. The patient administers 1-2 drops of an ophthalmic composition containing 0.1% w/v 8CPT-2Me-cAMP formulated in an aqueous solution to each eye twice daily. The treatment is expected to result in a noticeable improvement in ocular function.

As used in this specification and the appended claims, the singular forms “a,” “an,” and “the” include plural referents unless the content clearly dictates otherwise. Thus, for example, reference to a composition containing “a polynucleotide” includes a mixture of two or more polynucleotides. It should also be noted that the term “or” is generally employed in its sense including “and/or” unless the content clearly dictates otherwise. All publications, patents and patent applications referenced in this specification are indicative of the level of ordinary skill in the art to which this invention pertains. All publications, patents and patent applications are herein expressly incorporated by reference to the same extent as if each individual publication or patent application was specifically and individually indicated by reference. In case of conflict between the present disclosure and the incorporated patents, publications and references, the present disclosure should control.

It also is specifically understood that any numerical value recited herein includes all values from the lower value to the upper value, i.e., all possible combinations of numerical values between the lowest value and the highest value enumerated are to be considered to be expressly stated in this application. For example, if a concentration range is stated as 1% to 50%, it is intended that values such as 2% to 40%, 10% to 30%, or 1% to 3%, etc., are expressly enumerated in this specification. If a concentration range is “at least 5%, it is intended that all percentage values up to and including 100% are also expressly enumerated. These are only examples of what is specifically intended.

The invention has been described with reference to various specific embodiments and techniques. However, it should be understood that many variations and modifications may be made while remaining within the spirit and scope of the invention.

We claim:

1. A method of testing an agent for antiangiogenic activity comprising contacting an endothelial cell with the agent and assaying for activation of Rap-1 in the endothelial cell, activation of Rap-1 in the endothelial cell being indicative of antiangiogenic activity.

2. The method of claim 1, further comprising contacting a second endothelial cell with the agent and detecting cell flattening.

3. The method of claim 1, wherein the endothelial cell is a human microvascular endothelial cell or a human umbilical vascular endothelial cell.

4. The method of claim 1, wherein activation of Rap-1 is assayed by evaluating expression of Rap-1 guanine nucleotide exchange factors (GEFs) in the cell or performing a pull-down assay for activated Rap-1.

5. The method of claim 1, further comprising contacting a population of endothelial cells with the agent and assaying for antiangiogenic activity in the population of endothelial cells.

6. The method of claim 5, wherein assaying for antiangiogenic activity comprises evaluating chemotaxis in the population of endothelial cells.

7. The method of claim 5, wherein assaying for antiangiogenic activity comprises evaluating tubule formation in the population of endothelial cells.

8. The method of claim 1, wherein the agent is a cAMP analog.

9. A method of inhibiting angiogenesis in a cell population comprising contacting one or more of the cells in the population with an agent that activates Rap-1 or the Rap-1 signaling pathway.

10. The method of claim 9, wherein the agent is edema toxin.

11. The method of claim 9, wherein the agent is 8CPT-2Me-cAMP, or an analog thereof.

12. The method of claim 9, wherein the agent is a constitutively active Rap-1.

13. The method of claim 9, wherein the agent is identified by the method of claim 1.

14. The method of claim 9, wherein the cell population comprises an in vitro cell population.

15. The method of claim 9, wherein the cell population comprises an in vivo cell population.

16. The method of claim 15, wherein the cell population comprises two or more endothelial cells within a solid tumor.

17. The method of claim 15, wherein the agent is administered intratumorally.

18. The method of claim 16, wherein the agent is administered to the vasculature of the solid tumor.

19. The method of claim 16, wherein the microvessel density of the tumor is reduced.

20. The method of claim 16, wherein the tumor volume is reduced.

21. The method of claim 15, wherein the cell population comprises two or more endothelial cells in an ocular tissue.

22. The method of claim 21, wherein the cell population comprises two or more retinal or subretinal endothelial cells.

23. The method of claim 21, wherein the agent is administered topically to the eye, intravitrally or periocularly.

24. A method of treating a condition characterized by ocular neovascularization in a subject in need thereof comprising administering to the subject a therapeutically effective amount of an agent that activates Rap-1.

25. The method of claim 24, wherein the condition is selected from the group consisting of macular degeneration, diabetic retinopathy, retinopathy of prematurity, corneal graft rejection, neovascular glaucoma, retrolental fibroplasias, epidemic keratoconjunctivitis, Vitamin A deficiency, contact lens overwear, atopic keratitis, superior limbic keratitis, pterygium keratitis sicca, rubeotic glaucoma, and interstitial keratitis.

26. The method of claim 24, wherein the agent is a cAMP analog.

27. The method of claim 26, wherein the cAMP analog is 8CPT-2Me-cAMP.

28. The method of claim 27, wherein the 8CPT-2Me-cAMP is administered intravitrally, periocularly or topically to an eye of the subject.

29. The method of claim 24, wherein the agent is a polynucleotide encoding a constitutively active Rap-1 polypeptide.

30. An pharmaceutical composition comprising a Rap-1 activator.

31. A method of inhibiting angiogenesis in a cell population comprising delivering to at least one cell of the cell population a polynucleotide encoding a constitutively active Rap-1 polypeptide, the polynucleotide operably connected to a promoter functional in the at least one cell to obtain expression of the Rap-1 polypeptide at a level sufficient to inhibit angiogenesis.

32. The method of claim 31, wherein the cell population is located in a mammalian subject.

33. The method of claim 31, wherein the polynucleotide is comprised within a vector.

34. The method of claim 33, wherein the vector is a liposome.

35. The method of claim 34, wherein the vector is a viral vector.

36. The method of claim 35, wherein the viral vector is selected from the group consisting of an adenoviral vector, an adeno-associated viral vector, a vaccinia viral vector, a retroviral vector, a pox viral vector and a herpesviral vector.

37. A kit for inhibiting angiogenesis comprising a polynucleotide encoding a constitutively active Rap-1 polypeptide.

38. A method of inhibiting chemotaxis in a cell comprising contacting one or more cells with an agent that inhibits Rho kinase.
